(19) United States
(12) Patent Application Publication
(10) Pub. No.: US 2010/0143889 A1
(43) Pub. Date: Jun. 10, 2010

(54) RHABDOVIRIDAE VIRUS PREPARATIONS

(75) Inventors: Mark J. Federspiel, Rochester, MN (US); Troy R. Wegman, Rochester, MN (US); Kirsten K. Langfield, Lake City, MN (US); Henry J. Walker, Rochester, MN (US); Sharon A. Stephan, Rochester, MN (US)

Correspondence Address: FISH & RICHARDSON P.C., PO BOX 1022, MINNEAPOLIS, MN, 55440-1022, US

(73) Assignee: Mayo Foundation for Medical Education and Research

(21) Appl. No.: 12/628,789

(22) Filed: Dec. 1, 2009

Related U.S. Application Data
(60) Provisional application No. 61/119,291, filed on Dec. 2, 2008.

(51) Int. Cl. C12Q 1/70; C12N 7/00; C12N 7/02

(52) U.S. Cl. 435/5; 435/235.1; 435/239

(57) ABSTRACT

This document involves methods and materials related to obtaining Rhabdoviridae virus preparations. For example, methods and materials for obtaining large volume, high titer, high purity preparations of Rhabdoviridae viruses (e.g., VSV) with low or non-existent levels of contaminants are provided.

### CROSS-REFERENCE TO RELATED APPLICATIONS

This application claims the benefit of U.S. Provisional Application Ser. No. 61/119,291, filed Dec. 2, 2008, which is hereby incorporated by reference in its entirety.

### BACKGROUND

1. Technical Field

This document relates to methods and materials involved in obtaining Rhabdoviridae virus preparations.

2. Background Information

Rhabdoviridae viruses are a family of enveloped viruses having a single negative-strand of genomic RNA. The viruses within this family have a genome ranging from about 10 to 12 kilobases, which contains a minimum of five genes. Vesicular Stomatitis Virus (VSV) is a member of the Rhabdoviridae family and is categorized in the genus Vesicularvirus. VSV is bullet shaped, and has a genome that is about 11 kb. VSV replicates in the cytoplasm of infected cells, does not undergo genetic recombination or reassortment, has no known transforming potential, and does not integrate any part of its genome into the host (Barber, Viral Immunology, 17:516-527 (2004)).

The VSV genome encodes five polypeptides: nucleocapsid (N), non-structural protein (NS), matrix protein (M), glycoprotein (G), and a RNA-dependent RNA polymerase (L). All these polypeptides are synthesized from the genomic template as monocistronic, capped, polyadenylated mRNAs.

### SUMMARY

This document relates to methods and materials involved in Rhabdoviridae virus preparations and the production of Rhabdoviridae virus preparations. For example, this document provides methods and materials that can be used to obtain large volume, high titer, high purity preparations of Rhabdoviridae viruses (e.g., VSV) with levels of contaminants in compliance with federal regulations (e.g., levels that are acceptable to the Food and Drug Administration). Methods and materials described herein may be used, for example, to produce clinical quality Rhabdoviridae virus from cells (e.g., HEK293 cell cultures). Such preparations can have broad uses in research, development, and medical fields. For example, such preparations can allow medical professionals to deliver efficiently large quantities of Rhabdoviridae virus to patients for various clinical treatments (e.g., in virotherapy of cancer).

This document also relates to methods and materials involved in collecting enveloped Rhabdoviridae viruses (e.g., VSV) without disrupting their viral envelopes. These methods and materials can allow Rhabdoviridae viruses to retain their ability to infect host cells. Rhabdoviridae viruses can be collected from infected cells and effectively separated from the cells and cellular materials. The methods and materials provided herein can be used to process a large number of cells and/or a large volume of cell supernatants such that large quantities of Rhabdoviridae viruses are collected. In some cases, a preparation of Rhabdoviridae viruses can be obtained as described herein without using anion exchange. In some embodiments, the compositions provided herein can be sterile. For example, each step of a method used to make a virus preparation can be performed under sterile conditions. In some embodiments, good manufacturing practices (GMP) can be used to make the Rhabdoviridae virus preparations provided herein.

In general, one aspect of this document features a method for producing Rhabdoviridae viruses at titers of at least 10⁸ TCID₅₀ per mL directly in cell culture supernatants. The method comprises, or consists essentially of: (a) growing producer cells in serum-free medium to a cell density, (b) infecting the cells at a low M.O.I. in an original volume or after increasing cell density, (c) incubating the infected cells to produce an infected cell culture supernatant, and (d) harvesting the infected cell culture supernatant. The method can comprise supplementing the culture volume with fresh culture media after step (b). The incubating step (c) can be between 24 and 72 hours.

In another aspect, this document features a method for making a composition comprising Rhabdoviridae virus (e.g., a vesicular stomatitis virus), wherein the composition has a volume greater than 300 mL and a Rhabdoviridae virus titer greater than 10⁸ TCID₅₀ per mL. The method comprises, or consists essentially of: (a) obtaining a sample of Rhabdoviridae virus in serum-free medium, and (b) obtaining Rhabdoviridae virus from the sample to form the composition without performing an anion exchange step. The serum-free medium can have a volume between 20 mL and 200 L. The composition can have a Rhabdoviridae virus titer between 10⁸ TCID₅₀ per mL and 10¹⁶ TCID₅₀ per mL. The virus in step (a) can be replicated in a 293 cell. The method can comprise after step (a), contacting the sample with an enzyme. The enzyme can be an endonuclease. The step (b) can comprise performing a filtering step to remove the Rhabdoviridae virus from a non-Rhabdoviridae component in the sample. The step (b) can comprise a tangential flow filtering step. The Rhabdoviridae virus can be a vesicular stomatitis virus comprising nucleic acid encoding a human interferon β polypeptide.

In another aspect, this document features a method for assessing a composition comprising Rhabdoviridae viruses for the presence of non-Rhabdoviridae viruses. The method comprises, or consists essentially of, (a) contacting the composition with an antibody preparation under conditions wherein the preparation neutralizes the Rhabdoviridae viruses within the composition thereby forming a neutralized Rhabdoviridae virus sample, (b) incubating the sample with viable cells, and (c) determining whether or not the cells exhibit a cytopathic effect, wherein the presence of the cytopathic effect indicates that the composition comprises non-Rhabdoviridae viruses, and wherein the absence of the cytopathic effect indicates that the composition lacks non-Rhabdoviridae viruses. The Rhabdoviridae viruses can be vesicular stomatitis viruses, and the non-Rhabdoviridae viruses can be non-vesicular stomatitis viruses. The Rhabdoviridae viruses can be vesicular stomatitis viruses comprising nucleic acid encoding a human interferon β polypeptide. The antibody preparation can comprise polyclonal antibodies directed against a vesicular stomatitis virus and a monoclonal antibody directed against G-protein of a vesicular stomatitis virus. The cells can be mammalian cells (e.g., Vero cells). The incubating step can be performed for greater than eight days (e.g., eight to 30 days).

Unless otherwise defined, all technical and scientific terms used herein have the same meaning as commonly understood by one of ordinary skill in the art to which this invention pertains. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description, and from the claims.

### DESCRIPTION OF DRAWINGS

FIG. 1 is a schematic of a recombinant virus (VSV-IFNβ).

FIG. 2 is a graph plotting the titer for four different viral infections protocols.

FIG. 3 is a flowchart summarizing steps that can be used to produce a Rhabdoviridae virus preparation.

FIG. 4 is a diagram of steps that can be used to produce a Rhabdoviridae virus preparation.

FIG. 5 is a graph plotting the level of neutralization observed at the indicated days post infection for the indicated virus dilutions. The undiluted VSV sample contained about 9.3×10⁸ TCID₅₀ units per well. The undiluted and serially diluted VSV samples were treated with 475 μg of the anti-VSV monoclonal antibody.

### DETAILED DESCRIPTION

This document provides methods and materials related to producing Rhabdoviridae virus preparations. The Rhabdoviridae virus preparations can be large volume, high titer preparations of Rhabdoviridae virus. The preparations can be purified such that the Rhabdoviridae virus preparation is substantially free of non-viral polypeptides and host cell DNA. In some cases, a preparation can contain DNA. Such DNA can be treated such that most, if not all (e.g., at least 80, 85, 90, 95, or 99 percent), is less than 500 bp (e.g., less than 450, 400, 350, 300, 250, 200, 150, 100 bp) in length.

The preparations can have any volume. For example, the volume can be greater than 1 μL, greater than 1 mL, greater than 500 mL, greater than 1 L, greater than 100 L, greater than 1000 L, or greater than 10,000 L. In some embodiments, the volume can be less than 10,000 L, less than 1000 L, less than 100 L, less than 1 L, or less than 1 mL. In some embodiments, the preparation can have a volume that ranges from about 5 mL to about 500 L (e.g., from about 10 mL to about 250 mL, from about 25 mL to about 200 L, from about 50 mL to about 100 L, from about 300 mL to about 50 L, from about 10 L to about 50 L, from about 15 L to about 30 L, or from about 300 mL to about 25 L).

The preparation can have a Rhabdoviridae virus titer that is, for example, greater than 10¹, 10², 10³, 10⁴, 10⁵, 10⁶, 10⁷, 10⁸, 10⁹, 10¹⁰, 10¹¹, 10¹², 10¹³, 10¹⁴, or 10¹⁵ TCID₅₀ per mL. In some cases, the titer can be less than 10¹⁶, 10¹⁵, 10¹⁴, 10¹³, 10¹², 10¹¹, 10¹⁰, 10⁹, 10⁸, 10⁷, 10⁵, 10⁴, or 10³ TCID₅₀ per mL. It follows that the titer can also be between any of these concentrations. For example, the titer can be between 10⁵ and 10⁷ TCID₅₀ per mL, between 10⁵ and 10⁸ TCID₅₀ per mL, between 10⁶ and 10¹⁰ TCID₅₀ per mL, between 10⁷ and 10¹² TCID₅₀ per mL, between 10⁹ and 10¹² TCID₅₀ per mL, between 10¹⁰ and 10¹¹ TCID₅₀ per mL, or between 10² and 10¹² TCID₅₀.

The preparation can have any concentration of host cell DNA. For example, the preparation can contain less than 1 ng, 20 ng, 100 ng, 500 ng, 1 μg, 20 μg, or 100 μg of host cell DNA per mL. In some embodiments, the concentration of host cell DNA can be between 0 ng per mL and 10 mg per mL, between 2 and 250 ng per mL, between 10 and 300 ng per mL, between 40 and 500 ng per mL, between 500 ng and 5 μg per mL, between 0 ng per mL and 5 ng per mL, between 0 ng per mL and 10 ng per mL, or between 4 μg and 9 mg per mL. In some cases, the preparation can be substantially free of host cell DNA.

The preparation can have any concentration of non-viral polypeptides. For example, the preparation can contain less than 1 ng, 50 ng, 500 ng, 950 ng, 200 μg, 300 μg, 500 μg, 750 μg, or 1 mg of non-viral polypeptides per mL. In some cases, the concentration of non-viral polypeptides can range from 1 ng per mL to 10 mg per mL (e.g., from about 5 ng per mL to about 250 ng per mL, from about 500 ng per mL to about 50 μg per mL, or from about 20 ng per mL to about 1 mg per mL). In some embodiments, the preparation can be substantially free of non-viral polypeptides.

The viruses in a Rhabdoviridae preparation can be any type of Rhabdoviridae virus. For example, the preparation can contain a member of the Vesiculovirus genus (e.g., VSV) or a member of the Lyssavirus genus (e.g., Rabies virus), or any other type of Rhabdoviridae virus.

In some cases, the Rhabdoviridae virus can have a wild-type genome. In some cases, the preparation can contain genetically modified Rhabdoviridae viruses. The genetically modified Rhabdoviridae virus can have any nucleic acid sequence. For example, a Rhabdoviridae virus can have a hybrid genome, partially deleted genome, a genome with one or more point mutations, or a recombinant genome. A Rhabdoviridae virus having a partially deleted genome can have any segment of its genome deleted. In one embodiment, there can be complete or partial deletions of a Rhabdoviridae virus's N, NS, M, G, or L gene, or any other sequence found in a Rhabdoviridae virus genome. A Rhabdoviridae virus having one or more point mutations can have one or more nucleic acids inserted, deleted, or substituted at any position in its genome. For example, there can be one or more point mutations in a Rhabdoviridae virus's N, NS, M, G, or L genes, or any other sequence found in a Rhabdoviridae virus genome.

A recombinant genome can contain any nucleic acid sequence. For example, it can have one or more heterologous sequences at the 5′ end, the 3′ end, or anywhere between the 5′ and 3′ ends. A recombinant Rhabdoviridae virus genome can encode one or more antigens, tags, or non-viral polypeptides. For example, a recombinant Rhabdoviridae virus genome can contain nucleic acid encoding a human interferon β polypeptide, a GFP polypeptide, a human sodium iodide symporter polypeptide, or any other polypeptide.

Any method can be used to make a Rhabdoviridae virus preparation. For example, the methods provided herein can be used to make a Rhabdoviridae virus preparation having a high titer, large volume, and a high degree of purity. The viruses of such preparations can have stable envelopes. Typically, the preparation can be made by first infecting cells with a Rhabdoviridae virus at a low multiplicity of infection (MOI). However, any appropriate MOI can be used. For example, a MOI less than about 0.001, 0.01, 0.1, 0.5, 1.0, 2.0, 20, or 50 viruses per cell can be used. An MOI greater than 0.0001, 0.001, 0.01, 0.1, 0.5, 1.0, 2.0, 20, or 50 viruses per cell also can be used. In some embodiments, an MOI between about 0.01 and 50 viruses per cell can be used (e.g., an MOI between about 0.01 and 1 virus per cell, an MOI between about 0.5 and 10 viruses per cell, or an MOI between about 1 and 50 viruses per cell). The Rhabdoviridae virus used to infect the cells can come from any source. In some embodiments, Rhabdoviridae viruses can be rescued from a plasmid DNA molecular clone. In some cases, Rhabdoviridae viruses can be from a virus seed stock, a virus bank, a clinical quality virus preparation, an inoculated medium, a supernatant from an infected cell culture, or an infected cell.

Any type of cell can be infected with a Rhabdoviridae virus in order to replicate virus. For example, HEK293 cells, Vero cells, BHK21 cells, HeLa-S3 cells, or combinations thereof can be used. In some cases, wild-type or genetically modified cells can be used. For example, untransfected, transiently transfected, or stably transfected cells can be infected with Rhabdoviridae virus.

Following infection, the cells can be cultured under any appropriate condition for any appropriate length of time. In some embodiments, the cells can be cultured for more than 1 hour, 1 day, 2 days, 3 days, 4 days, 5 days, one week, two weeks, or one month. The cells can be cultured in any appropriate culture container (e.g., dishes, multi-well dishes, plates, flasks, microcarriers, roller bottles, or tubes). In some cases, a WAVE Reactor System 20/50 (GE Healthcare Bioscience Bioprocess Corp., Somerset, N.J.) can be used to culture the cells. The cells can be cultured at any temperature between about 27° C. and 39° C. For example, the cells can be cultured between about 27° C. and 37° C., between about 27° C. and 30° C., between about 29° C. and 32° C., or between about 30° C. and 37° C. The cells are cultured at CO₂ concentrations recommended by the media manufacturer.

The cells can be cultured in any appropriate type of medium. For example, the cells can be cultured in Ex-Cell 293 media+6 mM Glutamax, 293-SFMII media+4 mM Glutamax, DMEM media+5% fetal bovine serum (FBS)+4 mM Glutamax, VP-SFM media+2% FBS+4 mM Glutamax, or VP-SFM+4 mM Glutamax. The cells can be cultured in a mixture of two or more types of media. The cells can be cultured with or without serum in the medium. For example, the medium can contain no serum. In some cases, the cells can be cultured with glutamine or GlutaMAX™ (cell culture media that contains a stabilized form dipeptide from L-glutamine, L-alanyl-L-glutamine, that prevents degradation and ammonia build-up even during long-term cultures). In some embodiments, the medium can contain less than 0.1, 1, 5, 10, or 20 percent serum. In some cases, the medium can contain greater than 0.01, 2, or 15 percent serum. For example, the medium can contain between about 0.001 and 10 percent, between about 0.01 and 5 percent, between about 0.01 and 1 percent, or between about 0.01 and 0.1 percent serum.

After the cells infected with Rhabdoviridae virus are cultured under culture conditions for a desired length of time, any appropriate method can be used to collect the Rhabdoviridae viruses. In some embodiments, the viruses can be collected from infected cell lysate or supernatant in which infected cells were grown. Any appropriate method can be used to separate Rhabdoviridae viruses from cellular materials contained within a lysate or supernatant. In some embodiments, a lysate or supernatant can be pre-filtered and/or centrifuged to remove cells and debris. Pre-filtering can be performed using any appropriate method of pre-filtering. For example, a supernatant containing Rhabdoviridae virus can be filtered using a series of filters of sequentially decreasing pore size (e.g., the first filter may have a pore size between 40-8 microns, followed by a 5-3 micron filter, followed by a 1.5-0.2 micron filter). If the lysate or supernatant is centrifuged, the centrifuge can spin at a force between 500 g and 2000 g. The centrifuge can spin at any appropriate temperature (e.g., between about 0.5° C. and 14° C., between about 10° C. and 39° C., between about 12° C. and 29° C., or between about 17° C. and 39° C.). In some cases, a cell culture supernatant can be centrifuged at 675 g for 10 minutes at 4° C., and the resulting supernatant can be filtered through a series of filters (e.g., an 3-8 micron filter followed by a 1.5-0.2 micro filter).

Once Rhabdoviridae viruses have been separated from cellular materials, some residual cellular DNA and non-viral polypeptides can contaminate the sample containing the Rhabdoviridae viruses. Any appropriate method can be used to remove cellular DNA from the sample containing Rhabdoviridae viruses. For example, enzymes can be used to digest the DNA. These enzymes can be any DNA nucleases including, without limitation, endonucleases (e.g., Benzonase®; Merck) or exonucleases. In some cases, the DNA can be digested such that any remaining DNA is less than about 500, 450, 400, 350, 300, 250, 200, 150, or 100 bp in length. In some cases, about 10 to 20 units of Benzonase® per mL can be added to a solution containing Rhabdoviridae viruses to remove cellular DNA. The enzyme reactions can be carried out for any appropriate time and temperature (e.g., 1 hour at 15-40° C. followed by at least 24 hours at 2-8° C. in any appropriate container (e.g., a Bioprocess Bag obtained from Hyclone, Stedim, TC Tech)).

After digesting DNA, the solution containing Rhabdoviridae viruses can be filtered. For example, a tangential flow filter technique can be performed using hollow-fiber cartridges from GE Healthcare or Spectrum Laboratories. The cartridges may be made of polysulfone or polyethersulfone with a nominal pore size of 20 nM or 500,000 Daltons, or 50 nM or 750,000 Daltons. Tangential flow filtration can be used to concentrate the Rhabdoviridae viruses present in the sample. In some cases, a final filtration step can be performed using a standard filter (e.g., 0.2 or 0.45 or 1.2 micron filter). Any filter material can be used (e.g., polyethersulfone, PVDF, nylon, polypropylene). Once this final filtration step is completed, the sample can be aseptically vialed for use in clinical settings.

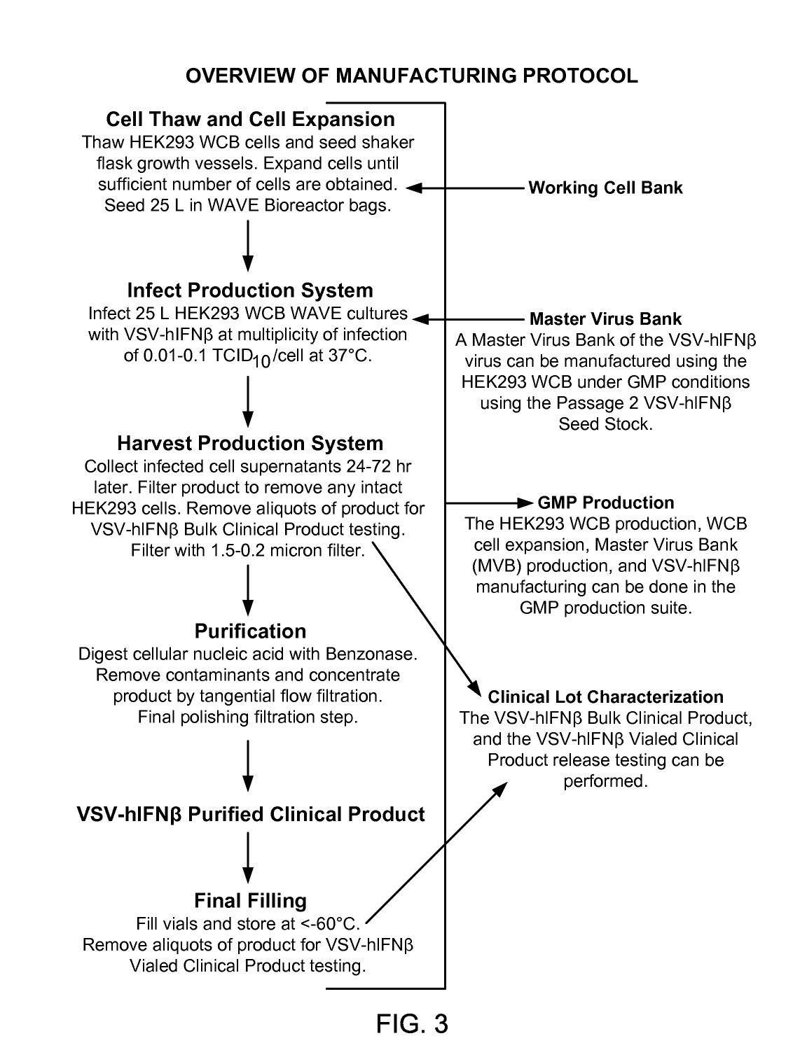

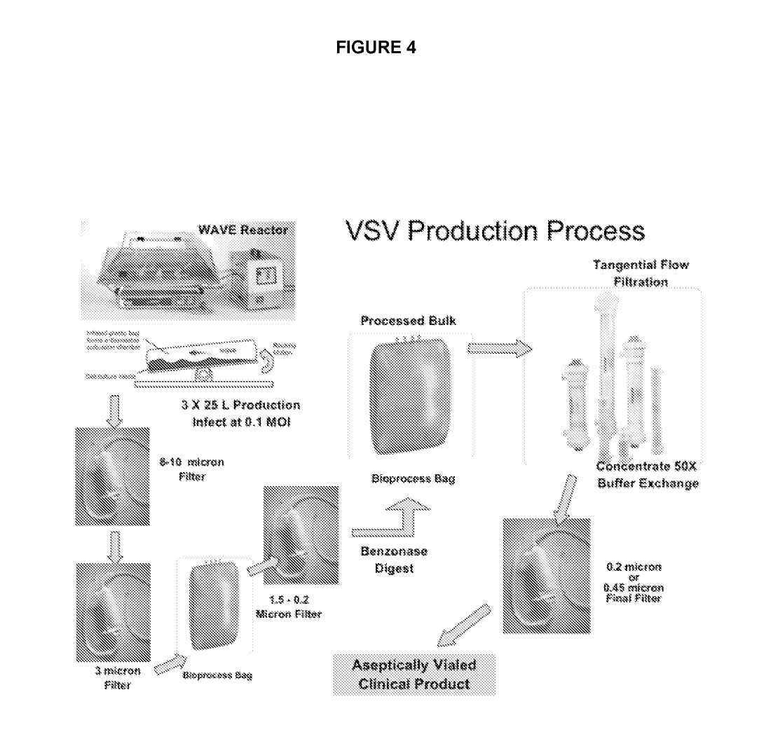

In some cases, the manufacturing and VSV production processes set forth in FIGS. 3 and 4 can be used.

In some cases, a composition containing Rhabdoviridae viruses (e.g., compositions containing VSV) can be analyzed for adventitious viruses (e.g., non-Rhabdoviridae viruses) before being released for use (e.g., clinical use). For example, a sample of a composition containing Rhabdoviridae viruses can be treated with anti-Rhabdoviridae virus antibodies to neutralize the ability of the Rhabdoviridae viruses to infect and/or replicate within cells. The anti-Rhabdoviridae virus antibodies can be polyclonal antibodies, monoclonal antibodies, or combinations thereof. For example, a combination of goat anti-VSV polyclonal antibodies and a monoclonal antibody directed against a G protein of VSV (e.g., the monoclonal antibody produced by a hybridoma having ATCC Accession No. CRL-2700) can be used to neutralize VSV. Once the desired Rhabdoviridae viruses (e.g., VSV) are neutralized, the sample can be incubated with viable cells. Examples of viable cells that can be used to assess a composition for adventitious viruses include, without limitation, Vero, MRC-5, Hs68, A549, HeLa, and NIH3T3 cells. After an incubation period (e.g., 4, 5, 6, 7, 8, 9, 10, 11, 12, 13, 14, 15, 20, 25, 30, or more days), the cells can be examined for evidence of a cytopathic effect or non-Rhabdoviridae virus replication. The presence of a cytopathic effect or non-Rhabdoviridae virus replication can indicate that the composition contains adventitious viruses (e.g., non-Rhabdoviridae viruses). The absence of a cytopathic effect or non-Rhabdoviridae virus replication can indicate that the composition lacks adventitious viruses (e.g., non-Rhabdoviridae viruses).

The invention will be further described in the following examples, which do not limit the scope of the invention described in the claims.

### EXAMPLES

### Example 1

### VSV-hIFNβ and the Production of VSV-hIFNβ Preparations

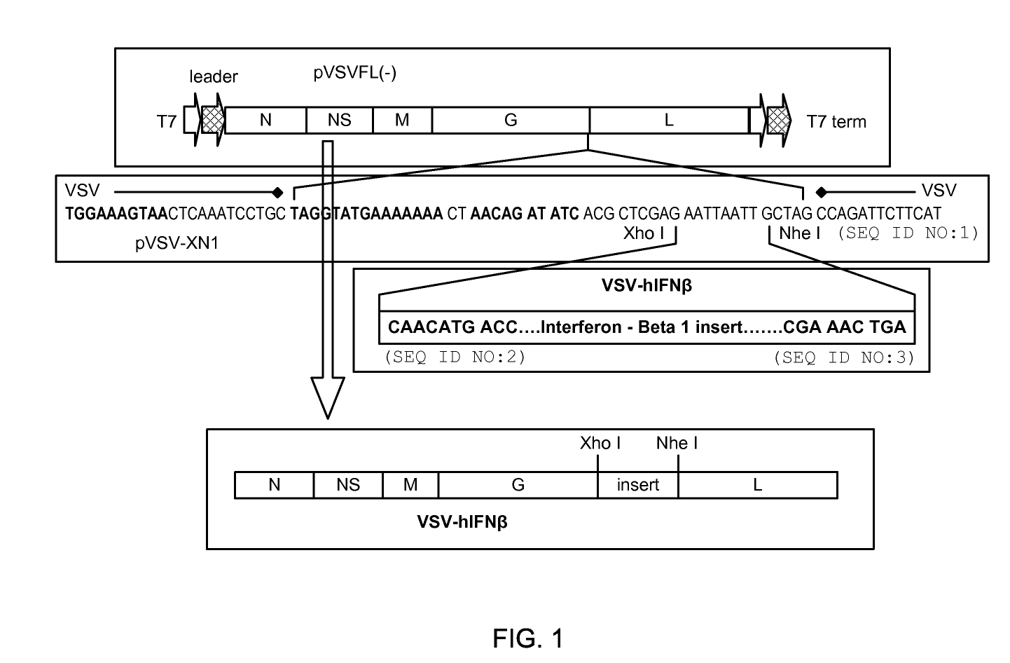

A recombinant VSV (VSV-hIFNβ; FIG. 1) was produced to contain nucleic acid encoding a human interferon beta 1 polypeptide that was inserted between the VSV-G and VSV-L genes as described elsewhere (Obuchi et al., J. Virology, 77:8843-8856 (2003); Schnell et al., J. Virol., 70:2318-2323 (1996); Lawson et al., Proc. Nat'l. Acad. Sci. USA, 92:4477-4481 (1995); Gallione et al., J. Virol., 39:529-535 (1981); Rose and Gallione, J. Virol., 39:519-534 (1981); and Schubert et al., Proc. Nat'l. Acad. Sci. USA, 82:7984-7988 (1985)).

The virus was plaque purified three times in a pre-clinical VERO working cell bank derived from a GMP master cell bank (BioReliance WHO VERO MCB p139). The VERO cells were adapted to serum-free medium (VPSFM and L-glutamine). The virus was amplified for one passage in VERO cells and called Passage 1 VSV-hIFNβ. A pre-clinical passage 2 VSV-hIFNβ Seed Stock, and a Passage 3 VSV-hIFNβ Preclinical Master Virus Bank (MVB) were produced using HEK293 suspension pre-clinical WCB cells and ExCell 293 serum-free media. Using this Preclinical MVB, a pre-clinical VSV-hIFNβ product (passage 4) was made using HEK293 suspension pre-clinical WCB cells and ExCell 293 serum-free media. The nucleotide sequence of the recombinant plasmid sequence encoding VSV-hIFNβ used to rescue the virus was determined and compared to the nucleotide sequence of pre-clinical passage 3 VSV-hIFNβ Preclinical MVB (Tables 1 and 2).

TABLE 1
Location of genes present in VSV
(Indiana Strain)* and VSVpcMVB.

| | |
|---|---|
| | VSVpcMVB |
| VSV* | (VSV-hIFNβ) |

| Gene | start | end | start | end |
|---|---|---|---|---|
| N | 64 | 1332 | 64 | 1332 |
| NS | 1396 | 2193 | 1396 | 2193 |
| M | 2250 | 2939 | 2250 | 2939 |
| G | 3078 | 4613 | 3078 | 4613 |
| hIFN-β 1 | Not present | Not present | 4666 | 5229 |
| L | 4733 | 11062 | 5339 | 11668 |

*(Locus NC_001560, Genbank Accession No. AC_001560, Genbank GI: 9627229).

TABLE 2
Comparison of the nucleotide sequence of the VSV
Indiana Strain to the VSV cloning plasmid.

| | | | VSV-hIFNβ | | |
|---|---|---|---|---|---|
| Nucleotide | VSV (Indiana)* | | Plasmid | | |
| position in | | Amino acid | | Amino acid | Amino Acid |

| | VSV-hIFNβ | Base | codon | Base | codon | | VSV | VSV-hIFNβ |
|---|---|---|---|---|---|---|---|---|
| Gene | Plasmid | found | used | found | used | position | Indiana | Plasmid |
| N | 103 | A | ATA | G | GTA | 14 | I | V |
| NS | 1614 | T | GGT | C | GGC | 73 | G | G |
| NS | 1625 | A | CAG | C | CCA | 77 | Q | P |
| | 1626 | G | | A | | 77 | Q | P |
| NS | 1724 | C | CCA | A | CAG | 110 | P | Q |
| | 1725 | A | | G | | 110 | P | Q |
| M | 2646 | A | ACT | G | GCT | 133 | T | A |
| M | 2918 | T | GAT | C | GAC | 224 | D | D |
| M | 2925 | A | AGC | G | GGC | 227 | S | S |
| G | 3246 | A | ATA | T | TTA | 57 | I | L |
| G | 3365 | G | AGT | T | ATT | 96 | Q | H |
| hIFNβ^{#} | 4818 | | | | | | | |
| hIFNβ^{#} | 5171 | | | | | | | |
| L | 5458 | T | AAT | C | AAC | 40 | N | N |
| L | 5459 | T | TTG | C | CTG | 41 | L | L |
| L | 5497 | G | TTG | A | TTA | 53 | L | L |
| L | 5521 | G | CCG | A | CCA | 61 | P | P |
| L | 5575 | A | ACA | G | ACG | 79 | T | T |
| L | 5597 | T | TCA | C | CCA | 87 | S | P |
| L | 7404 | C | ACT | G | AGT | 691 | T | S |
| L | 11415 | C | ACT | T | ATT | 2026 | T | I |

*(Locus NC_001560, Genbank Accession Number AC_001560, Genbank GI: 9627229).
^{#}Homo sapiens interferon, beta 1, Locus EF064725, Genbank Accession Number EF064725.1, Genbank GI: 1176065601

No differences were found in the polypeptide coding regions of the VSV genes (N, NS, M, G, and L) when comparing the original constructed plasmid to the passage 3 VSV-hIFNβ-Preclinical MVB.

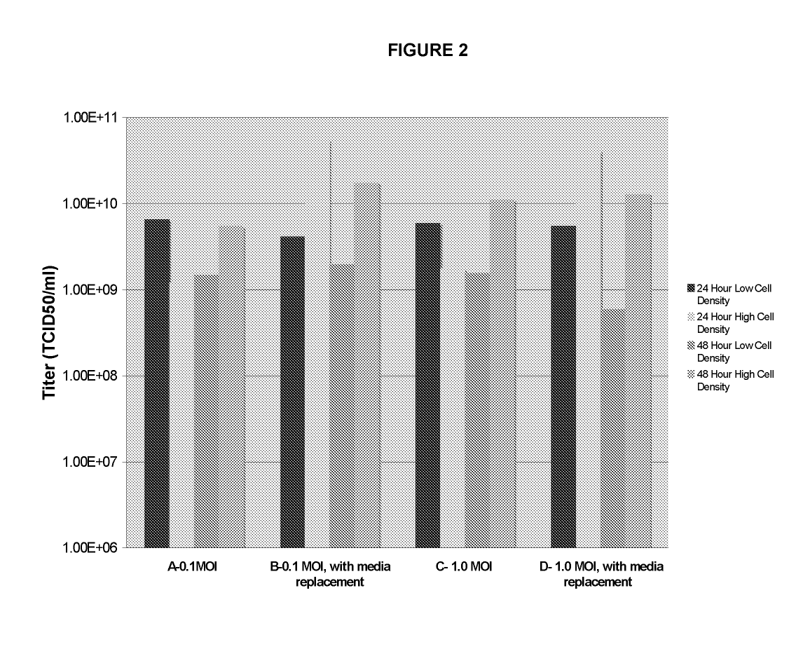

Vials of cells of a GMP certified HEK293 Suspension Master Cell Bank (MCB) were obtained from the NIH National Cancer Institute for the GMP manufacture of a GMP WCB and clinical virus products. Infection conditions were evaluated using HEK293 SFS cells and VSV-hIFNβ to identify optimal conditions (FIG. 2).

### Example 2

### VSV Preparation Yields

HEK293 SFS cells were grown in shake flasks in Ex Cell 293 media supplemented with 6 mM Glutamax. In general, an average cell density of 3.85×10⁶ viable cells/mL and viability of 99% was used for the infection procedure on Day 0. The titers of unprocessed bulk and final purified vialed products were determined (Table 3).

TABLE 3
VSV-hIFNβ titers.

| | | | Titer of | Titer of Final Purified |
|---|---|---|---|---|
| | | | Unprocessed | Vialed Product After |
| | | | Bulk | Freeze/Thaw TCID₅₀ |
| Name of Virus | Name of Prep | Scale | TCID₅₀ units/mL | units/mL |
| VSV human IFN beta | VSV human IFN beta | 250 mL | 3.43 × 10¹⁰ | NA |
| | P2-Pre-clinical | 1 × 1 L shake flask | | |
| VSV human IFN beta | VSV human IFN beta | 500 mL | 2.0 × 10¹⁰ | NA |
| | P3- Pre-clinical MVB | 2 × 1 L shake flask | | |
| VSV human IFN beta | VSV human IFN beta | 2 L | 3.72 × 10¹⁰ | 2.4 × 10¹¹ |
| | xxxx-01 | 2 × 2.8 L shake flask | | |
| | P4 - pre-clinical | | | |
| VSV human IFN beta | VSV human IFN beta | 25 L | 2.4 × 10⁸ | 4.37 × 10⁸ |
| | xxxx-08 | 1 × 50 L wave bag | | |
| | P4 - pre-clinical | | | |
| VSV human IFN beta | Preclinical 0.01 MOI | 1 L | 7.41 × 10¹⁰ | Not Purified |
| | Harvest 24 h | 1 × 2 L wave bag | | |
| VSV human IFN beta | Preclinical 0.1 MOI | 1 L | 3.98 × 10¹⁰ | Not Purified |
| | Harvest 24 h | 1 × 2 L wave bag | | |
| VSV mouse IFN beta | VSV mouse IFN beta | 2 L | 1.42 × 10¹⁰ | 6.61 × 10⁹ |
| | yyyy-01 | 2 × 2 L wave bags | | Note: Major loss on final |
| | p4 - pre-clinical | | | filter |
| VSV mouse IFN beta | VSV mouse IFN beta | 2 L | 1.31 × 10⁹ | Not final filtered |
| | yyyy-02 | 2 × 2 L wave bags | | |
| | p4 - pre-clinical | | | |
| VSV mouse IFN beta | VSV mouse IFN beta | 500 mL | 1.89 × 10⁹ | 9.85 × 10¹⁰ |
| | yyyy-03 | 2 × 1 L shake flask | | |
| | p4 - pre-clinical | | | |
| VSV mouse IFN beta | VSV mouse IFN beta | 2 L | Not done | 2.45 × 10¹⁰ |
| | xxxx-01 | 2 × 2.8 L flask | | |
| | p4 - pre-clinical | | | |
| VSV mouse IFN beta | VSV mouse IFN beta | 2 L | 3.02 × 10¹⁰ | 2.24 × 10¹¹ |
| | xxxx-02A <4.6 psi | 2 × 2.8 L flask | | |
| | p4 - pre-clinical | | | |
| VSV mouse IFN beta | VSV mouse IFN beta | 1.8 L | 1.26 × 10¹⁰ | 1.15 × 10¹¹ |
| | xxxx-02B >4.6 psi | 2 × 2.8 L flask | | |
| | p4 - pre-clinical | | | |
| VSV rat IFN beta | VSV rat IFN beta | 50 ml | 1.0 × 10¹¹ | NA |
| | P2 - pre-clinical | 1 × 250 mL shake flask | | |
| VSV rat IFN beta | VSV rat IFN beta | 500 ml | 3.47 × 10¹⁰ | NA |
| | P3 - pre-clinical MVB | 2 × 1 L shake flask | | |
| VSV rat IFN beta | VSV rat IFN beta | 2 L | 6.03 × 10¹⁰ | 4.17 × 10¹¹ |
| | xxxx-01 | 2 × 2.8 L shake flask | | |
| | P4 - pre-clinical | | | |
| VSV GFP | VSV GFP | 50 ml | 3.02 × 10¹⁰ | NA |
| | P2- Pre-Clinical | 1 × 250 mL shake flask | | |
| VSV GFP | VSV GFP | 500 ml | 3.72 × 10¹⁰ | NA |
| | P3- Pre-Clinical | 2 × 1 L shake flask | | |
| VSV GFP | VSV GFP | 2 L | 1.35 × 10¹⁰ | 2.19 × 10¹⁰ |
| | P4- Pre-Clinical | 2 × 2.8 L shake flask | | |

### Example 3

### Pilot 2 L Prep and Purification of VSV Human IFN Beta xxxx-01

HEK293 SFS cells were grown in shake flasks in Ex Cell 293 media supplemented with 6 mM Glutamax. A total of 2 L of culture at an average cell density of 3.85×10⁶ viable cells/mL and viability of 99% was used for the infection procedure on Day 0.

On the day of infection, the entire 2 L cell culture was centrifuged at 2500 rpm (673 g)×10 minutes in an SLC 4000 rotor (Sorvall). The cell pellets were resuspended in a total volume of 600 mL of the conditioned media from the original culture. The total volume was divided as follows: 300 ml per 2.8 L shake flask, shaking at 70-75 rpm, 37 C, 5% CO₂. Virus was added at an MOI of 0.01 and incubated with the resuspended cells for 1 hour under the same incubation conditions. After the initial infection period, each culture was brought to a 1 L volume with fresh culture media (Ex Cell 293+6 mM Glutamax) and incubation continued under the same conditions for approximately 24 hours.

At approximately 24 h after infection the entire 2 L of infected culture was harvested by centrifuging at 2500 rpm (673 g)×10 minutes in an SLC 4000 rotor (Sorvall). The cell pellets were discarded and the cleared cell supernatant was processed further.

The cleared cell supernatant was filtered sequentially through a 1500 cm² 3 micron Versapor filter [Pall], followed by a 200 cm², 0.2 micron polyethersulfone filter (Supor, Mini-Kleenpak, Pall). Following filtration, at least 20,000 units/L of Benzonase and 2 mL/L of a 1 M solution of Magnesium Chloride were added to the cleared cell supernatant. At this point the cleared cell supernatant is renamed processed bulk product. In this example, the processed bulk product was incubated at room temperature for about 1 hour then at 2-8 C for about 24 hours prior to further purification.

Purification was performed using tangential flow filtration using a 1050 cm² Spectrum 20 nm (500 kD) polyethersulfone cartridge. The TFF conditions used: shear rate approx 3000 sec⁻¹ and the transmembrane pressure less than 3 psig. The 2 L of processed bulk was concentrated five fold then diafiltered against 5 volumes of sucrose buffer (5% sucrose, 2 mM magnesium chloride, 50 mM Tris-HCl pH 7.4). Following diafiltration, the retentate was concentrated about 30 fold to a final volume of 63 mL. The final retentate was then filtered through a 200 cm², 0.2 micron polyethersulfone filter (Supor, Mini-Kleenpak, Pall). The filtered final retentate was vialed as the Purified Product and frozen at <=−65° C.

The volume, protein content, titer, and DNA content was determined for each step (Table 4).

TABLE 4
Analysis of VSV preparations.

| VSV human | | | | | | | | | | |
|---|---|---|---|---|---|---|---|---|---|---|
| IFN beta xxxx- | | | Total | | | Total | % TCID50 | | | |
| 01, p4, 2nd | Volume | Protein | Protein | % Protein | TCID50 | TCID50 | units | DNA | Total | % DNA |
| isolate. | (mL) | (ug/mL) | (mg) | Remaining | units/mL | units | remaining | (ng/mL) | DNA (ug) | remaining |
| Unprocessed | 2000 | 2532 | 5064 | 100.0 | 3.72E+10 | 7.44E+13 | 100.0 | 11232.00 | 22464.00 | 100.0 |
| Bulk Product | | | | | | | | | | |
| [Cleared cell | | | | | | | | | | |
| supernatant] | | | | | | | | | | |
| Cleared cell | 1920 | 2588 | 4969 | 98.1 | 1.20E+10 | 2.304E+13 | 31.0 | 11281.50 | 21660.48 | 96.4 |
| supernatant, | | | | | | | | | | |
| post 3.0 um filter | | | | | | | | | | |
| Cleared cell | 1900 | 2548 | 4841 | 95.6 | 7.41E+10 | 1.4079E+14 | 189.2 | 11424.50 | 21706.55 | 96.6 |
| supernatant, | | | | | | | | | | |
| post 0.2 um filter | | | | | | | | | | |
| Processed Bulk | 1900 | 2637 | 5010 | 98.9 | 1.02E+11 | 1.938E+14 | 260.5 | 2051.00 | 3896.90 | 17.3 |
| Product, Pre- | | | | | | | | | | |
| TFF | | | | | | | | | | |
| Permeate | 4000 | 1354 | 5416 | 107.0 | ND | ND | ND | 480.00 | 1920.00 | 8.5 |
| Final | 63 | 1971 | 124 | 2.5 | 3.63E+12 | 2.29E+14 | 307.4 | 678.50 | 42.75 | 0.2 |
| retentate, [~30X] | | | | | | | | | | |
| Final | 42 | 1712 | 72 | 1.4 | 1.72E+12 | 7.224E+13 | 97.1 | 683 | 28.69 | 0.1 |
| retentate, [30X], | | | | | | | | | | |
| Post 0.2 um | | | | | | | | | | |
| filter. [Not | | | | | | | | | | |
| frozen]¹ | | | | | | | | | | |
| Final | 42 | 1712 | 72 | 1.4 | 2.40E+11 | 1.008E+13 | 13.5 | 683 | 28.69 | 0.1 |
| retentate, [30X], | | | | | | | | | | |
| Post 0.2 um | | | | | | | | | | |
| filter. [Post- | | | | | | | | | | |
| freeze/thaw].^{2,3} | | | | | | | | | | |

### Example 4

### Pilot 25 L Prep and Purification of VSV Human IFN Beta xxxx-08

HEK293 SFS cells were grown in a 50 L WAVE Bioreactor with perfusion filter in Ex Cell 293 media supplemented with 6 mM Glutamax, rocking at 15-20 rpm, 37° C., 5% CO₂. A total of 25 L of culture at an average cell density of 3.21×10⁶ viable cells/mL and viability of 99% was used for the infection procedure on Day 0.

On the day of infection, media was removed reducing the final volume from 25 L to 7.5 L. Virus was added at an MOI of 0.01 and incubated with the concentrated cells for 1 hour under the same incubation conditions. After the initial infection period, the culture was brought back to 25 L volume with fresh culture media (Ex Cell 293+6 mM Glutamax) and incubation continued under the same conditions.

At approximately 24 hours after infection the entire 25 L of infected culture was harvested by first filtering through a 10 Inch, 10 micron Kleenpak Nova Prefilter Capsule (Pall), followed by a 10 Inch, 3 micron Kleenpak Nova Prefilter Capsule (Pall), followed by a 10 Inch, 0.2 micron polyethersulfone filter Supor, Kleenpak Nova Capsule (Pall). Following filtration, at least 10,000 units/L of Benzonase and 2 mL/L of a 1 M solution of Magnesium Chloride were added to the filtered cell supernatant. At this point the filtered cell supernatant is renamed processed bulk product. In this example, the processed bulk product was incubated at room temperature for about 1 hour then at 2-8° C. for about 120 hours prior to further purification.

Purification was performed using tangential flow filtration using a 16,000 cm² Spectrum 20 nm (500 kD) polyethersulfone cartridge. The TFF conditions used: shear rate approx 3000-4000 sec⁻¹ and the transmembrane pressure less than 5 psig. The 23.5 L of processed bulk was concentrated five fold then diafiltered against approximately 7-volumes of sucrose buffer (5% sucrose, 2 mM magnesium chloride, 50 mM Tris-HCl pH 7.4). Following diafiltration, the retentate was concentrated about 65 fold to a final volume of 350 mL. The final retentate was then filtered through a 0.15 m², 0.2 micron polyethersulfone (Supor, Kleenpak Capsule, Pall). The filtered final retentate was vialed as the Purified Product and frozen at <=−65° C. The volume, protein content, titer, and DNA content was determined for each step (Table 5).

TABLE 5
Analysis of VSV preparations.

| | | | | | | | % TCID50 | | | |
|---|---|---|---|---|---|---|---|---|---|---|
| | | | | | | | units | | | |
| | | | | | | | remaining | | | |
| | | | | | | | [Relative | | | |
| VSV human | | | Total | | | Total | to | | Total | |
| IFN beta xxxx- | Volume | Protein | Protein | % Protein | TCID50 | TCID50 | Processed | DNA | DNA | % DNA |
| 08, p4. | (mL) | (ug/mL) | (mg) | Remaining | units/mL | units | Bulk] | (ng/mL) | (ug) | remaining |
| Wave pre- | 24000 | ND | ND | ND | 2.40E+08 | 5.76E+12 | | TBD | ND | ND |
| harvest sample | | | | | | | | sample | | |
| [contains cells] | | | | | | | | contains | | |
| | | | | | | | | cells | | |
| Wave harvest, | 23900 | ND | ND | ND | 2.24E+08 | 5.3536E+12 | | 10891.00 | 260294.90 | ND |
| post 10 micron | | | | | | | | | | |
| filter [contains | | | | | | | | | | |
| residual cells] | | | | | | | | | | |
| Cleared cell | 23800 | 2453 | 58381 | 100.0 | 1.07E+08 | 2.5466E+12 | | 6285.00 | 149583.00 | 100.0 |
| supernatant, | | | | | | | | | | |
| post 3 micron | | | | | | | | | | |
| filter [This | | | | | | | | | | |
| would be | | | | | | | | | | |
| equivalent to | | | | | | | | | | |
| an official bulk | | | | | | | | | | |
| product for | | | | | | | | | | |
| GMP testing]. | | | | | | | | | | |
| Cleared cell | 23500 | 2487 | 58445 | 100.1 | 4.47E+07 | 1.05045E+12 | | 5450.00 | 128075.00 | 85.6 |
| supernatant, | | | | | | | | | | |
| post 0.2 micron | | | | | | | | | | |
| filter | | | | | | | | | | |
| Processed Bulk | 23500 | 2573 | 60466 | 103.6 | 4.79E+07 | 1.12565E+12 | 100.0 | 985.00 | 23147.50 | 15.5 |
| Product, Pre- | | | | | | | | | | |
| TFF | | | | | | | | | | |
| Permeate | 58000 | 1085 | 62930 | 107.8 | 2.10E+05 | 12.2E+10 | 1.1 | 398.00 | 23084.00 | 15.4 |
| Final | 350 | 2388 | 836 | 1.4 | 2.04E+09 | 7.14E+11 | 63.4 | 1101.00 | 385.35 | 0.3 |
| retentate, [~65X] | | | | | | | | | | |
| Final | 325 | ND | ND | ND | ND*** | ND | ND | ND | ND | ND |
| retentate, [~65X], | | | | | | | | | | |
| Post 0.2 um | | | | | | | | | | |
| filter. [Not | | | | | | | | | | |
| frozen]* | | | | | | | | | | |
| Purified Pre- | 305 | 1625 | 496 | 0.8 | 4.37E+08 | 1.33285E+11 | 11.8 | 924.5 | 281.97 | 0.2 |
| Clinical Product, | | | | | | | | | | |
| Passage 4, | | | | | | | | | | |
| Vialed and | | | | | | | | | | |
| frozen** | | | | | | | | | | |

Note 1:
Wave reactor volume was 25 L but 1 L of cell slurry was left in the Wave bag.
**Characterization run on this material after freeze/thaw
***Titer data not available on filtered retentate prior to freeze/thaw because initial assay was out of range.

### Example 5

### Neutralization of VSV Prior to In Vitro or In Vivo Testing for Adventitious Viruses

VSV products (e.g. bulk products or clinical products) are analyzed for adventitious viruses before being released for clinical use. Prior to testing for adventitious viral contaminants, VSV virus are neutralized by VSV-specific anti sera. The following describes conditions for neutralizing VSV.

Bulk product from VSV Master Virus Bank (MVB) and/or VSV Clinical Product production batches can have a titer of 2-3e10 TCID₅₀ units/mL. The bulk products are essentially virus containing cell culture supernatant that has been cleared of cells. The reagents and supplies are as follows: (1) VSV-specific antisera (e.g., goat anti-VSV hIFNb); (2) monoclonal antibody (MAb) (e.g., CRL2700 anti-VSV G protein [1.89 mg/mL, current batch]); (3) tissue culture plates, 6 well; (4) fetal bovine serum; (5) DMEM Gibco#11965-126 with Pen/Strep; (6) VERO cells (BioReliance WHO Magenta VERO); (7) D-PBS without Mg⁺⁺ or Ca⁺⁺; (8) trypsin-EDTA Gibco #25200; (9) 2×DMEM; (10) 5 mL polypropylene tubes; (11) sterile, disposable serological pipettes; and (12) sterile disposable pipette tips.

Cocalico Biologicals, Inc. was contracted to produce goat and rabbit anti-VSV polyclonal antibody preparations. UV inactivated VSV-hIFNB was supplied and used as the immunogen to produce the polyclonal antibodies to VSV. Goats and rabbits were immunized with the inactivated virus preparation. Briefly, goats received a primary immunization with 300 μg inactivated VSV, followed by 5 subsequent booster injections each containing 150 μg. The periods between injections varied from 1 week to 1 month. The immunogen was homogenized with Freund's adjuvant, and the animals were injected subcutaneously and intramuscularly. Rabbits were immunized in a similar manner but dosing was less. The primary immunization contained 100 μg, and subsequent boosters contained 50 μg. Up to five boosters were used to maximize the titer of neutralization antibody. Sera were tested for VSV neutralization activity after the 3^{rd}, 4^{th}, and 5^{th} injections. Exsanguination of goats yielded about 1100 mL, while exsanguination of rabbits yielded about 100 mL.

The monoclonal antibody was produced from a commercially available hybridoma cell line (ATCC Accession No. CRL-2700; cell line name: I1-Hybridoma). The cell line is a mouse hybridoma that produces a mouse IgG2a antibody specific for the major surface glycoprotein (G-protein) of VSV (Indiana Serotype) (Lefrancios et al., Virology, 121: 157-167 (1982)). The hybridoma was adapted to grow in reduced serum media. The hybridoma was scaled up, and the cell supernatant was concentrated. The concentrated antibody was diafiltered into DMEM culture media. Several hundred milligrams of antibody were prepared at a final antibody concentration of approximately 1 mg/mL.

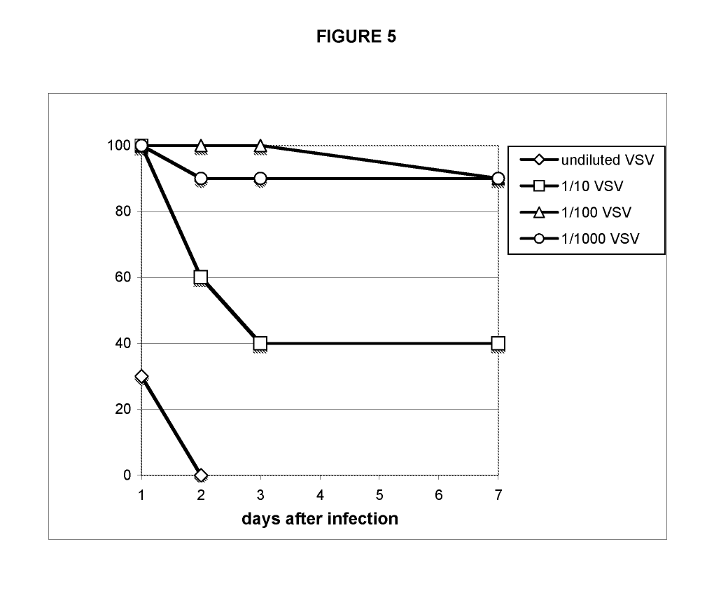

The following experiment was performed to test the ability of the monoclonal antibody to neutralize the vesicular stomatitis virus with human interferon beta gene insert (VSV humanIFNB). VERO cells were treated with undiluted VSV, which contained about 9.3×10⁸ TCID₅₀ units per well, as well as diluted VSV preparations. Both the undiluted and serially diluted VSV preparations were treated with 475 μg of the monoclonal antibody. Treatment with the monoclonal antibody alone resulted in only partial neutralization of VSV (FIG. 5).

Use of the goat sera in combination with the monoclonal antibody resulted in complete neutralization of VSV infection (Table 6). Goat sera alone resulted in only partial neutralization of the VSV infection (average of <50%) using standard adsorption times (Table 6). The pooled rabbit anti-sera neutralized the VSV infection of VSV-GFP (Table 7).

TABLE 6
Neutralization of VSV infection using goat polyclonal
anti-VSV antibodies supplemented with (or without)
monoclonal antibody (MAb) against VSV G-protein.

| | | % VSV Neutralization | |
|---|---|---|---|
| | amount of virus | with Goat Antisera | |
| Virus neutralized | (TCID50 units) | without MAb | with MAb |
| VSV mouseIFNB | 2.40E+07 | 0% | 100% |
| | 2.40E+06 | 12% | 100% |
| VSV GFP | 9.30E+07 | 19% | 100% |
| | 9.30E+06 | 44% | 100% |
| VSV humanIFNB | 5.00E+07 | 50% | in progress |
| VSV humanIFNB | 5.00E+07 | 100% | Not applicable |
| (With increased | | | |
| adsorption time) | | | |

TABLE 7
Neutralization of VSV infection (VSV GFP) using
rabbit antisera raised against inactivated VSV hIFNB.

| | | % VSV Neutralization with Pooled | | |
|---|---|---|---|---|
| | | Rabbit Antisera dilutions | | |
| Virus | amount of virus | Undiluted | | |
| neutralized | (TCID50 units) | sera | 1/10 sera | 1/100 sera |
| VSV GFP | 9.30E+07 | 100% | 0% | 0% |

This procedure was optimized using VERO cells grown in serum-containing media and subcultured with trypsin/EDTA using standard cell culture techniques. The viruses used to develop this protocol were pre-clinical Master Virus Bank, titers ranged from 2-3e10 TCID₅₀ units/mL.

The goat sera preparation was heat inactivated before use at 55° C. for 25 minutes and used undiluted, which significantly reduced toxicity of the sera without reducing its neutralization effect. All wells were treated individually to avoid cross contamination between wells.

Prior to the day of neutralization (Day 0), VERO cells were seed at 3×10⁵ cells/well in 2.0 mL of 5% FBS DMEM with pen/strep in a 6 well tissue culture plate and incubated for 24 hours at 37° C. Trypsin-EDTA was used to release the cells. For the adsorption step on day 0, the cells were about 50% confluent. In step 1, the pre-adsorption step, the following samples were prepared by combining the following reagents in a tube, mixing, and incubating at 37° C. on an orbital shaker at ˜100 rpm for 2 hours:

Negative control: 0.5 mL 5% FBS DMEM cultured media per well.

Positive Control: 0.05 mL VSV sample plus 0.45 mL 5% FBS DMEM with pen/strep per well.

Antibody control: 0.25 mL anti-VSV goat sera and 0.25 mL MAb plus 0.05 mL 5% FBS DMEM with pen/strep per well.

Neutralization samples: 0.05 mL of VSV sample plus 0.25 mL goat anti-VSV sera plus 0.25 mL MAb per well.

In step 2, the adsorption step at the end of the incubation, an equal volume of 2×DMEM was added to each tube. For example, 0.5 mL of 2×DMEM was added to the 0.5 mL mixture contained in tube. The 6 well plates containing the VERO cells were removed from the 37° C. incubator. The conditioned media were removed from the plate wells with a pipet. 0.9 mL of each respective test sample was added to each well of the 6 well plates, and the plates were incubated for 1 hour at 37° C.

In step 3, the plates were removed from incubator, and the test sample mixture was removed from each well separately to prevent cross-contamination. Each well was rinsed three times with 2 mL of serum free-DMEM. 2.5 mL of 5% FBS DMEM with pen/strep was added to each well, and the plates were incubated at 37° C., 5% CO₂.

On day 1 after initial adsorption, the test wells were refed with antisera in media. This step is referred to as backadding. The negative and antibody control wells were refed with media only. The positive control wells and any wells showing cytopathic effect (CPE) were not refed. Briefly, the test plates were removed from 37° C. The media were removed from all wells using a pipet, changing pipets between each well to prevent cross-contamination. Each neutralization test well not showing CPE was refed with 0.8 mL of a mixture of 1 part undiluted antibody to 1 part undiluted goat anti-sera to 2 parts 2×DMEM [for example: 0.25 mL Mab, 0.25 mL goat anti-sera, and 0.5 mL 2×DMEM]. The wells were incubated for 1 hour at 37° C., 5% CO₂. Then, the plates were removed from the incubator, the media was removed from each well, and the cells were refed with 2.5 mL of 5% FBS DMEM per well and incubated at 37° C., 5% CO₂.

On day 7 or 8, the media was removed from each well, and each well was rinsed with 2 mL of D-PBS without Ca or Mg. 0.5 mL of trypsin/EDTA was added, and the plates were incubated at 37° C. for 1 to 3 minutes. The plates were removed from incubator, and 0.5 mL 5% FBS DMEM was added to neutralize. 0.1 mL of cell suspension was transferred to a fresh well containing 2.5 mL of 5% FBS DMEM with pen/strep on a new 6 well plate. The new plates were incubated at 37° C., 5% CO₂.

The wells showing no CPE were refed every 3-4 days with 5% FBS DMEM with pen/strep and subcultured every 7 to 8 days until day 28.

Preparations with no adventitious viral contaminants exhibited no break-through of virus replication (CPE) in any of the neutralized wells. The positive control wells exhibited CPE at day 1-2. The negative control and antibody control wells exhibited no CPE and no death of cells due to anti-sera toxicity.

These results demonstrate the development of reagents and procedures capable of neutralizing VSV-containing samples so that the samples can be tested for the presence of adventitious viral contaminants.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method for producing Rhabdoviridae viruses at titers of at least 10⁸ TCID₅₀ per mL directly in cell culture supernatants, said method comprising:
(a) growing producer cells in serum-free medium to a cell density,
(b) infecting said cells with a Rhabdoviridae virus at a low M.O.I. in an original volume or after increasing cell density,
(c) incubating the infected cells to produce an infected cell culture supernatant, and
(d) harvesting said infected cell culture supernatant.

2. The method of claim 1, wherein said method comprises supplementing the culture volume with fresh culture media after step (b).

3. The method of claim 1, wherein said incubating step (c) is between 24 and 72 hours.

4. The method of claim 1, wherein said Rhabdoviridae virus is a vesicular stomatitis virus.

5. The method of claim 4, wherein said vesicular stomatitis virus comprises nucleic acid encoding a human interferon β polypeptide.

6. A method for making a composition comprising Rhabdoviridae virus, wherein said composition has a volume greater than 300 mL and a Rhabdoviridae virus titer greater than 10⁸ TCID₅₀ per mL, said method comprising:
(a) obtaining a sample of Rhabdoviridae virus in serum-free medium, and
(b) obtaining Rhabdoviridae virus from said sample to form said composition without performing an anion exchange step.

7. The method of claim 6, wherein said serum-free medium has a volume between 20 mL and 200 L.

8. The method of claim 6, wherein said composition has a Rhabdoviridae virus titer between 10⁸ TCID₅₀ per mL and 10¹⁶ TCID₅₀ per mL.

9. The method of claim 6, wherein the virus in step (a) was replicated in a 293 cell.

10. The method of claim 6, wherein said method comprises after step (a), contacting said sample with an enzyme.

11. The method of claim 6, wherein said enzyme is an endonuclease.

12. The method of claim 6, wherein said step (b) comprises performing a filtering step to remove said Rhabdoviridae virus from a non-Rhabdoviridae component in said sample.

13. The method of claim 6, wherein said step (b) comprises a tangential flow filtering step.

14. The method of claim 6, wherein said Rhabdoviridae virus is a vesicular stomatitis virus.

15. The method of claim 14, wherein said vesicular stomatitis virus comprises nucleic acid encoding a human interferon β polypeptide.

16. A method for assessing a composition comprising Rhabdoviridae viruses for the presence of non-Rhabdoviridae viruses, wherein said method comprises:
(a) contacting said composition with an antibody preparation under conditions wherein said preparation neutralizes said Rhabdoviridae viruses within said composition thereby forming a neutralized Rhabdoviridae virus sample,
(b) incubating said sample with viable cells, and
(c) determining whether or not said cells exhibit a cytopathic effect, wherein the presence of said cytopathic effect indicates that said composition comprises non-Rhabdoviridae viruses, and wherein the absence of said cytopathic effect indicates that said composition lacks non-Rhabdoviridae viruses.

17. The method of claim 16, wherein said Rhabdoviridae viruses are vesicular stomatitis viruses, and said non-Rhabdoviridae viruses are non-vesicular stomatitis viruses.

18. The method of claim 16, wherein said Rhabdoviridae viruses are vesicular stomatitis viruses comprising nucleic acid encoding a human interferon β polypeptide.

19. The method of claim 16, wherein said antibody preparation comprises polyclonal antibodies directed against a vesicular stomatitis virus and a monoclonal antibody directed against G-protein of a vesicular stomatitis virus.

20. The method of claim 16, wherein said cells are mammalian cells.

21. The method of claim 16, wherein said incubating step is performed for greater than eight days.


SEQUENCE LISTING (3 sequences)

SEQ ID NO: 1 - DNA, 84 bp, Vesicular stomatitis virus
tggaaagtaactcaaatcctgctaggtatgaaaaaaactaacagatatcacgctcgagaattaattgctagccagattcttcat

SEQ ID NO: 2 - DNA, 10 bp, Artificial Sequence
  5′-linker sequence for IFNBeta insert
caacatgacc

SEQ ID NO: 3 - DNA, 9 bp, Artificial Sequence
  3′-linker sequence for IFNBeta insert
cgaaactga